(19) United States
(12) Patent Application Publication
(10) Pub. No.: US 2008/0262010 A1
(43) Pub. Date: Oct. 23, 2008

(54) CRYSTALLINE POLYMORPHS OF N-(3-(DIMETHYLAMINO)PROPYL)-4-(4-(3-FLUORO-4-METHOXYPHENYL) PYRIMIDIN-2-YLAMINO) BENZENESULFONAMIDE AS D-GLUCORONATE SALTS

(75) Inventors: Mahmoud Mirmehrabi, Laval (CA); Abdolsamad Tadayon, Kirkland (CA); Subodh Deshmukh, White Plains, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/105,520

(22) Filed: Apr. 18, 2008

Related U.S. Application Data
(60) Provisional application No. 60/925,443, filed on Apr. 20, 2007.

(51) Int. Cl. A61K 31/505; C07D 239/42; A61P 35/00

(52) U.S. Cl. 514/275; 544/332

(57) ABSTRACT

The present invention relates to methods for preparing one or more crystalline polymorphs of a compound of formula I:

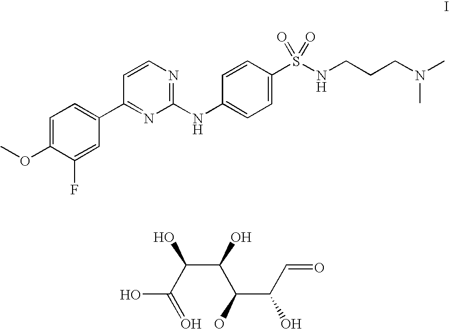

and structurally related compounds. The present invention is also directed to methods for converting one polymorph to other different polymorphs of formula I and structurally related compounds.

### CROSS REFERENCE TO RELATED APPLICATIONS

This application claims the benefit of priority under 35 U.S.C. §119(e) to U.S. Provisional Patent Application No. 60/925,443 filed on Apr. 20, 2007, which is hereby incorporated by reference in its entirety.

### FIELD OF THE INVENTION

The present invention relates to anilino-pyrimidine benzenesulfonamide analogs that are useful for inhibiting protein kinase activity. The invention is directed to methods for preparing and manufacturing certain crystalline forms and polymorphs of substituted anilino-pyrimidine benzenesulfonamides as pharmaceutically acceptable salts. In particular, the invention is directed to methods for preparing and manufacturing crystalline polymorphs of N-(3-(dimethylamino)propyl)-4-(4-(3-fluoro-4-methoxyphenyl)-pyrimidin-2-ylamino)benzenesulfonamide as D- and L-glucoronate salts.

### BACKGROUND OF THE INVENTION

Certain substituted anilino-pyrimidine benzenesulfonamide analogs have been discovered, which are useful for inhibiting protein kinase activity. One such compound is N-(3-(dimethylamino)propyl)-4-(4-(3-fluoro-4-methoxyphenyl)-pyrimidin-2-ylamino)benzenesulfonamide. The compound is prepared in the form of a free base, which has little to no water solubility.

The compound, in the form of a free base, hydrates, solvates or acid salts, belongs to a class of drugs typically referred to as IKK inhibitors. Nuclear factor-κB (NF-κB) is a transcriptional factor that regulates the expression of important genes related to cell survival. Aberrant expression of IKK has been correlated with activation of NF-κB and, in turn, tumorigenesis and cell proliferation. High IKK levels may also promote tumorigenesis by negatively regulating other transcription factors, such as FOXO factors. It has been described in Hu, M. (2004) “IκB Kinase Promotes Tumorigenesis through Inhibition of Forkhead FOXO3a,” Cell, 117, 225-237 and Haefner, B. (2002) “NF-κB: arresting a major culprit in cancer,” Drug Discovery Today, 7, 653-663. Thus, inhibiting IKK may inhibit cell proliferation and tumorigenesis.

Methods for synthesizing substituted anilino-pyrimidine benzenesulfonamides, such as N-(3-(dimethylamino)propyl)-4-(4-(3-fluoro-4-methoxyphenyl)pyrimidin-2-ylamino)benzenesulfonamide, is described in PCT Publication WO 2006/044457 A1 and U.S. Pat. No. 6,794,403.

The crystalline form of a particular drug as a salt, a hydrate, a solvate, and/or any polymorph thereof is often one important determinant of the drug's ease of preparation, stability, solubility, storage stability, ease of formulation and in-vivo pharmacology. Different crystalline forms of the same composition, known as polymorphs, occur when a composition crystallizes in different lattice arrangements or where solvent molecules including, but not limited to, water molecules are incorporated into the crystalline lattice, resulting in solids with different thermodynamic properties and stabilities specific to the particular form of the drug. It is entirely possible that one crystalline form is preferable over another where certain aspects such as ease of preparation, stability and like parameters are deemed to be critical. Similarly, greater solubility and/or superior pharmacokinetics may be desired characteristics.

Certain substituted anilino-pyrimidine benzenesulfonamide compounds have been shown to inhibit inappropriately high kinase activity, as disclosed in U.S. Pat. No. 6,048,866. One limitation of such anilino-pyrimidine benzenesulfonamide compounds is that they are not water soluble in a free base form. There is a need for crystalline, water-soluble forms of substituted anilino-pyrimidine benzenesulfonamide compounds that selectively inhibit kinase activity. The present compositions fulfill this need, including IKK inhibitors. Compositions of the invention are useful in the treatment of conditions including, but not limited to for example, polycystic kidney disease, colonic polyps, cancer, and stroke in mammals.

Because improved drug formulations showing, for example, better bioavailability or better stability are progressively achieved, there is an ongoing need for new or purer crystalline forms of existing drug molecules. Methods for preparing and manufacturing crystalline polymorphs of N-(3-(dimethylamino)propyl)-4-(4-(3-fluoro-4-methoxyphenyl)-pyrimidin-2-ylamino)benzenesulfonamide as D-glucoronate salts are described, characterized and claimed herein. In addition, methods for converting one polymorph to other different polymorphs are also disclosed and claimed herein.

### SUMMARY OF THE INVENTION

Accordingly, the invention provides a method for manufacturing crystalline forms and polymorphs of compounds of formula I:

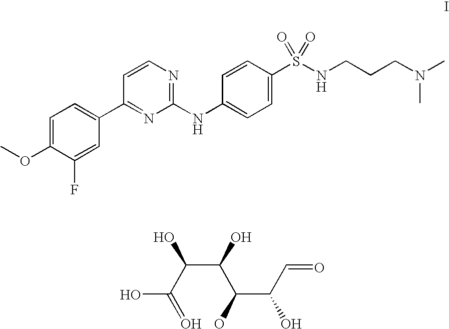

comprising the steps of: dissolving an amount of N-(3-(dimethylamino)propyl)-4-(4-(3-fluoro-4-methoxyphenyl)-pyrimidin-2-ylamino)benzenesulfonamide and D-glucoronic acid, as a mixture, in one or more solvents; and precipitating one crystalline polymorph of the compound of formula I as the D-glucoronate salt from the mixture.

The invention also provides a method for manufacturing crystalline polymorphs of compounds of formula I, comprising the step of: recrystallizing one crystalline polymorph of the compound of formula I, from one or more solvents, to precipitate a different crystalline polymorph of the compound of formula I. The pharmaceutically acceptable salt of the compound of formula I includes any pharmaceutically acceptable solvates and hydrates of the salt.

The invention also provides a method for manufacturing crystalline polymorphs of compounds of formula I, comprising the steps of: dissolving an amount of N-(3-(dimethylamino)propyl)-4-(4-(3-fluoro-4-methoxyphenyl)-pyrimidin-2-ylamino)benzenesulfonamide and D-glucoronic acid, as a mixture, in one or more solvents; precipitating one crystalline polymorph of the compound of formula I as the D-glucoronate salt from the mixture; and recrystallizing the precipitated one crystalline polymorph of the compound of formula I, from the one or more solvents by diluting with water or from a different one or more solvents, to precipitate a different crystalline polymorph of the compound of formula I. The pharmaceutically acceptable salt of the compound of formula I includes any pharmaceutically acceptable solvates and hydrates of the salt.

The invention also provides a method for converting one crystalline polymorph of a compound of formula I to one or more different polymorphs of the compound of formula I:

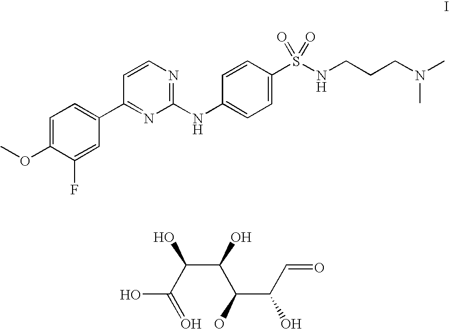

comprising the steps of: heating an amount of one polymorph of the compound of formula I to a temperature that converts the one crystalline polymorph of the compound of formula I to a different polymorph of the compound of formula I as the pharmaceutically acceptable salt. The new one or more crystalline polymorphs of the compound of formula I includes any pharmaceutically acceptable solvates and hydrates of the salt.

The invention also provides a method for converting one crystalline polymorph of a compound having formula I to one or more different polymorphs of the compound of formula I:

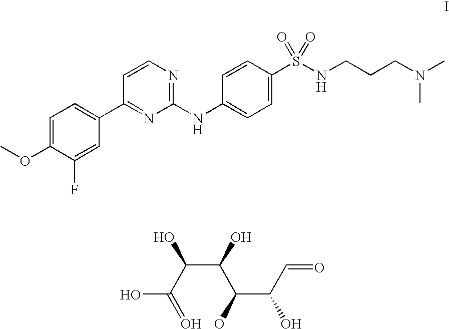

comprising the steps of: dissolving an amount of N-(3-(dimethylamino)propyl)-4-(4-(3-fluoro-4-methoxyphenyl)-pyrimidin-2-ylamino)benzenesulfonamide and D-glucoronic acid, as a mixture, in one or more solvents; precipitating one crystalline polymorph of the compound of formula I as the mono-D-glucoronate salt from the mixture; and recrystallizing an amount of the precipitated one crystalline polymorph of the compound of formula I, from the one or more solvents by diluting with water or from a different one or more solvents, while heating the mixture to convert the one crystalline polymorph of the compound of formula I to a different polymorph of the compound of formula I. The new one or more crystalline polymorphs of the compound of formula I includes any pharmaceutically acceptable solvates and hydrates of the salt.

The invention also provides methods for preparing and manufacturing pharmaceutically acceptable compositions comprising one or more of the crystalline forms or polymorphs of specific anilino-pyrimidine benzenesulfonamide compounds of formula I comprising the steps of: preparing a crystalline form or polymorph of the compound of formula I and adding one or more pharmaceutically acceptable additives or carriers.

### BRIEF DESCRIPTION OF THE FIGURES

FIG. 1 depicts a polycrystalline X-ray diffraction (XRD) pattern of one polymorph of N-(3-(dimethylamino)propyl)-4-(4-(3-fluoro-4-methoxyphenyl)pyrimidin-2-ylamino)-benzenesulfonamide as a D-glucoronate salt (Form I), where the diffraction angle (2θ) ranges from 0-30 degrees with a step of 0.01 degrees.

FIG. 2 depicts a differential scanning calorimetry (DSC) scan for N-(3-(dimethylamino)propyl)-4-(4-(3-fluoro-4-methoxyphenyl)pyrimidin-2-ylamino)benzenesulfonamide as a D-glucoronate salt (Form II).

FIG. 3 depicts a thermogravimetric analysis (TGA) thermogram for N-(3-dimethylamino)propyl)-4-(4-(3-fluoro-4-methoxyphenyl)pyrimidin-2-ylamino)benzenesulfonamide as a D-glucoronate salt (Form II).

FIG. 4 depicts a polycrystalline XRD pattern of a different polymorph of N-(3-(dimethylamino)propyl)-4-(4-(3-fluoro-4-methoxyphenyl)-pyrimidin-2-ylamino)benzenesulfonamide as a mono-D-glucoronate salt (Form II), where the diffraction angle (2θ) ranges from 0-30 degrees with a step of 0.01 degrees.

FIG. 5 depicts a polycrystalline XRD pattern of another different polymorph of N-(3-(dimethylamino)propyl)-4-(4-(3-fluoro-4-methoxyphenyl)pyrimidin-2-ylamino)-benzenesulfonamide as a D-glucoronate salt (Form II), where the diffraction angle (2θ) ranges from 0-30 degrees with a step of 0.01 degrees.

FIG. 6 depicts a polycrystalline XRD pattern of yet another different polymorph of N-(3-(dimethylamino)propyl)-4-(4-(3-fluoro-4-methoxyphenyl)pyrimidin-2-ylamino)-benzenesulfonamide as a D-glucoronate salt (Form IV), where the diffraction angle (2θ) ranges from 0-30 degrees with a step of 0.01 degrees.

### DETAILED DESCRIPTION OF THE INVENTION

For convenience, certain terms employed in the specification, examples, and appended claims are collected here.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In one embodiment, a straight chain or branched chain alkyl has 6 or fewer carbon atoms in its backbone. The term “alkyl” can be used alone or as part of a chemical name as in for example, “trialkylorthoformate”. The terms “alkenyl” and “alkynyl” refer to unsaturated aliphatic groups analogous in length and possible substitution to the alkyls described above, but which contain at least one double or triple carbon-carbon bond, respectively. Analogous substitutions can be made to alkenyl and alkynyl groups to produce, for example, alkenylamines, alkynylamines, alkenylamides, alkynylamides, alkenylimines, alkynylimines, thioalkenyls, thioalkynyls, carbonyl-substituted alkenyls or alkynyls, alkenoxyls, alkynoxyls, metalloalkenyls and metalloalkynyls.

The term “alkoxy” as used herein refers to an alkyl group, as defined above, having an oxygen radical attached thereto. Representative alkoxyl groups include methoxy, ethoxy, propyloxy, tert-butoxy and the like. The term alkoxy can be used alone or as part of a chemical name as in for example, “alkoxy-enaminonitrile”. Alkoxy also means a group —OR, wherein R is an alkyl, alkenyl, or alkynyl group which can optionally be substituted with one or more functional groups. Hydroxy means —OH. Carbonyl means carbon bonded to oxygen with a double bond, i.e., C═O. Amino means the —NH₂ group.

The term “aryl” as used herein includes 4-, 5-, 6-, 7- and 10-membered carbocyclic single ring or fused multiple ring aromatic groups, which may be substituted or unsubstituted. Accordingly the term “phenyl” refers to a 6-membered carbocyclic single ring, which is partially substituted with substituents and other chemical groups at positions 1-5. The term “heteroaryl” refers to a 4 to 10 aromatic membered ring structure, which ring structure includes one to four heteroatoms. Heteroaryls include, but are not limited to, pyrrolidine, oxolane, thiolane, piperidine, piperazine, pyrrole, furan, thiophene, imidazole, oxazole, thiazole, triazole, pyrazole, pyridine, pyrazine, pyridazine and pyrimidine, and morpholine. The term “heteroatom” as used herein means an atom of any element other than carbon or hydrogen. Suitable examples of heteroatoms include, but are not limited to for example, nitrogen, oxygen, sulfur, phosphorus, and selenium.

The term “halogen” refers to an atom of fluorine, chlorine, bromine, or iodine.

As used herein, the term “substituted” is contemplated to include all permissible substituents of organic compounds. Typically, suitable substituents of organic compounds include acyclic and cyclic, branched and unbranched, carbocyclic and heterocyclic, aromatic and nonaromatic substituents of organic compounds and inorganic substituents, such as halogen and amino. The substituents can be one or more and the same or different for appropriate organic compounds. For purposes of this invention, the heteroatoms such as nitrogen may have hydrogen substituents, halogen substituents, halogen substituents and/or any suitable or conventional substituents of organic compounds described herein which satisfy the valencies of the heteroatoms. This invention is not intended to be limited in any manner by the suitable substituents of organic compounds.

Hydrates are solid compounds containing water molecules combined in a definite ratio as an integral part of the crystalline compound. Examples of hydrates include, but are not limited to for example: hemihydrate, monohydrate, dihydrate, trihydrate, quadrahydrate, pentahydrate and hexahydrate. Hydrates also are intended to include solids compounds containing water molecules combined in a non-stoichiometric ratio as an integral part of the crystalline compound.

Solvates are solid compounds containing solvent molecules combined in a definite ratio as an integral part of the crystal. Solvates also are intended to include solids compounds containing solvent molecules combined in a non-stoichiometric ratio as an integral part of the crystalline compound.

The present invention is directed to methods for preparing and manufacturing crystalline forms and polymorphs of certain substituted anilino-pyrimidine benzenesulfonamide compounds in the form of corresponding pharmaceutically acceptable salts, pharmaceutical compositions including the crystalline forms and polymorphs as corresponding pharmaceutically acceptable salts, pharmaceutical formulations including the crystalline forms and polymorphs as corresponding pharmaceutically acceptable salts, and methods of converting one crystalline polymorph of a certain substituted aniline-pyrimidine benzenesulfonamide compound in the form of a pharmaceutically acceptable salt to one or more different polymorphs of the certain substituted aniline-pyrimidine benzenesulfonamide compound in the form of the corresponding pharmaceutically acceptable salt.

In one embodiment, the certain substituted aniline-pyridine benzenesulfonamide are provided as compounds of formula I:

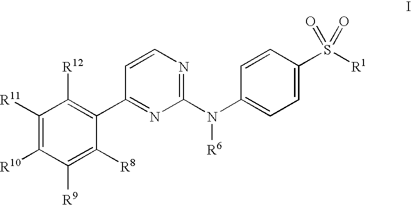

in the form of pharmaceutically acceptable salts wherein, R¹ is —NR²R³, wherein R² and R³ are independently selected from the group consisting of: C₁-C₅ substituted alkyl, C₂-C₅ substituted alkenyl, C₂-C₅ substituted alkynyl, C₂-C₅ substituted aryl or phenyl, C₁-C₅ substituted heteroaryl, hydroxyl, C₁-C₅ substituted alkoxy, C₁-C₅ substituted alkylamino, C₁-C₅ substituted arylamino, C₁-C₅ substituted heteroarylamino, —NCOR⁴, —COR⁴, —CONR²R³, SO₂R⁵, C₄-C₁₀ substituted 3 to 10 membered cyclic amines containing 0 to 3 heteroatoms; R⁴ and R⁵ are each selected from the group consisting of hydrogen, methyl, trifluoromethyl, substituted alkyl, substituted aryl, and substituted heteroaryl; R⁶ is selected from the group consisting of hydrogen, methyl, C₂-C₅ substituted alkyl, C₁-C₅ substituted alkylcarbonyl, and C₁-C₅ substituted alkoxycarbonyl; and wherein R⁸-R¹² are independently selected from the group consisting of: C₁-C₅ alkyl, F, Cl, Br, I, C₁-C₅ alkoxy, C₁-C₅ alkylamine, C₁-C₅ alkylamino, C₁-C₅ amide, C₁-C₅ ester, hydroxy, and C₁-C₅ alkyl-, C₁-C₅ alkoxy-, C₁-C₅ alkylamino-substituted amides, NH₂, trifluoromethyl, C₁-C₅ substituted alkyl trifluoromethyl, and phenyl. According to a separate embodiment, the pharmaceutically acceptable salts of the compounds of formula I include pharmaceutically acceptable solvates, and hydrates thereof.

In a separate embodiment, the certain substituted anilino-pyrimidine benzenesulfonamide compounds are provided as compounds formula II:

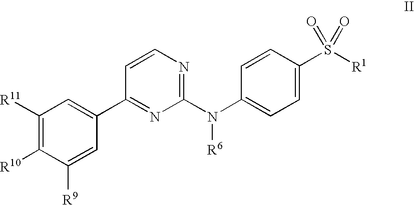

in the form of a pharmaceutically acceptable salt, wherein R¹, R⁶, and R⁹-R¹¹ are defined as above.

According to one embodiment, a pyrimidin-2-yl substituted phenyl group is provided, as described in U.S. Pat. No. 6,794,403, which is used as a substituent for the 2-position of the pyrimidine ring.

According to a separate embodiment, R¹⁰ is a para-substituted phenyl, an optionally substituted thienyl, and an optionally benzothiophene, wherein the optional substitution R⁸, R⁹, R¹⁰, R¹¹ and R¹² are independently at least one of C₁-C₅ alkyl, F, Cl, Br, C₁-C₅ alkoxy, amine, C₁-C₅ alkylamino, C₁-C₅ amide, C₂-C₅ ester, or hydroxy, and the C₁-C₅ alkyl, C₁-C₅ alkoxy, C₁-C₅ alkylamino, NH₂ and amide optionally substituted with at least one C₁-C₂ alkyl, C₁-C₄ alkoxy, amine, C₁-C₂ alkylamino, C₁-C₄ amide, C₂-C₄ ester, hydroxy, thienyl, or phenyl.

In one embodiment, the pyrimidin-2-yl substituted phenyl group is a multi-substituted phenyl group substituted at least at the para-position. In another embodiment, the pyrimidin-2-yl substituted phenyl group is a di-substituted phenyl group substituted at least at the para-position. In another embodiment, the pyrimidin-2-yl substituted phenyl group is a di-substituted phenyl group substituted at positions selected from the group consisting of 2,4-, 3,4-, 4,5-, and 4,6-positions, as described in U.S. Pat. No. 6,794,403, which is used a substituent for the 2-position of the pyrimidine ring.

Exemplary substituents for the pyrimidin-2-yl substituted phenyl group include, for example, C₁-C₅ alkyl, F, Cl, Br, I, C₁-C₅ alkoxy, amine, C₁-C₅ alkylamino, C₁-C₅ amide, C₂-C₅ ester, or hydroxy, and the alkyl, alkoxy, alkylamino, NH₂ and amide may optionally be substituted with at least one C₁-C₂ alkyl, C₁-C₄ alkoxy, amine, C₁-C₂ alkylamino, C₁-C₄ amide, C₂-C₄ ester, hydroxy, thienyl, or phenyl. Other exemplary substituents for R³ include, for example, alkoxy, trifluoromethyl, fluoro, hydroxy, and NR²R³ where R² is COR⁴ and R³ is hydrogen.

According to a separate embodiment, the pyrimidin-2-yl substituted phenyl group is a least di-substituted.

Alkylamino means the —NHR or NR where R is a C₁-C₄ alkyl group, which optionally may be substituted.

According to a separate embodiment, R¹ is selected from the group consisting of NR²R³, optionally substituted imidazolyl, and optionally substituted alkyl. In another embodiment, R¹ is NR²R³, and R² and R³ are independently selected from the group consisting of hydrogen, alkyl, amino and alkylamino (including cyclic amines), alkylhydroxy, alkanoyl, alkoxy, alkoxycarbonyl, carbonyl, carboxyl, aralkyl, optionally substituted phenyl, heteroaryl, and COR⁴ where R⁴ is alkyl or aralkyl. In yet another embodiment, R¹ is NH₂, -(dimethylamino)ethyl, or -(dimethylamino)propyl.

In another embodiment of R¹, R² and R³ are taken together to form an optionally substituted 3 to 12 membered monocyclic or bicyclic ring containing 0 to 4 heteroatoms. In one embodiment, R¹ is an optionally substituted 5 to 6 membered heterocyclic group containing at least one nitrogen atom and 0 to 1 additional heteroatoms. R¹ can be, for example, an optionally substituted morpholinyl group, an optionally substituted piperazinyl group, or an optionally substituted pyrrolidinyl group.

In another embodiment, R¹ is NR²R³, and R¹ is selected from the group of structures listed as Set 2a:

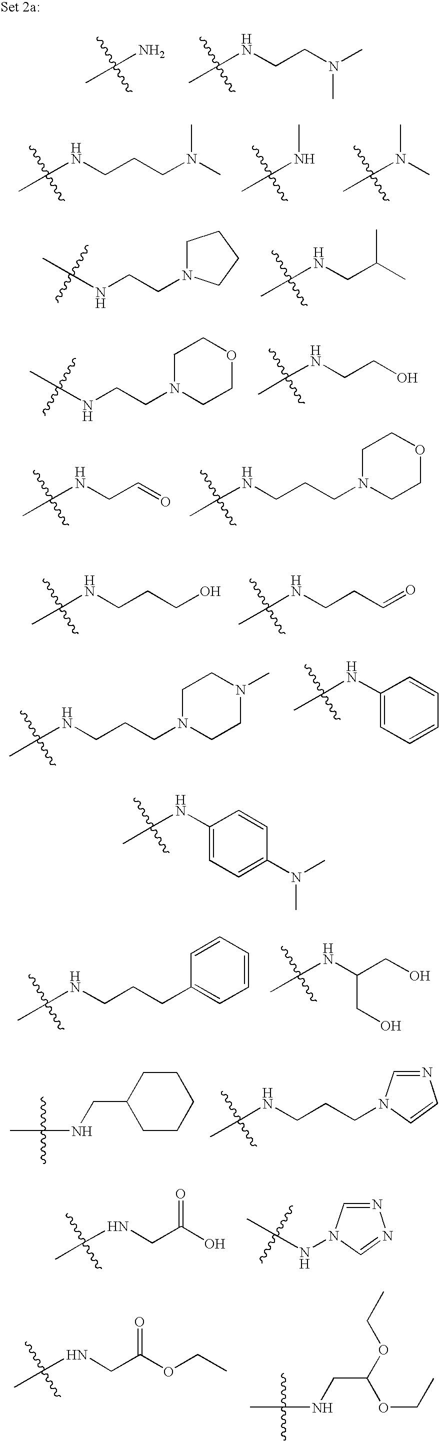

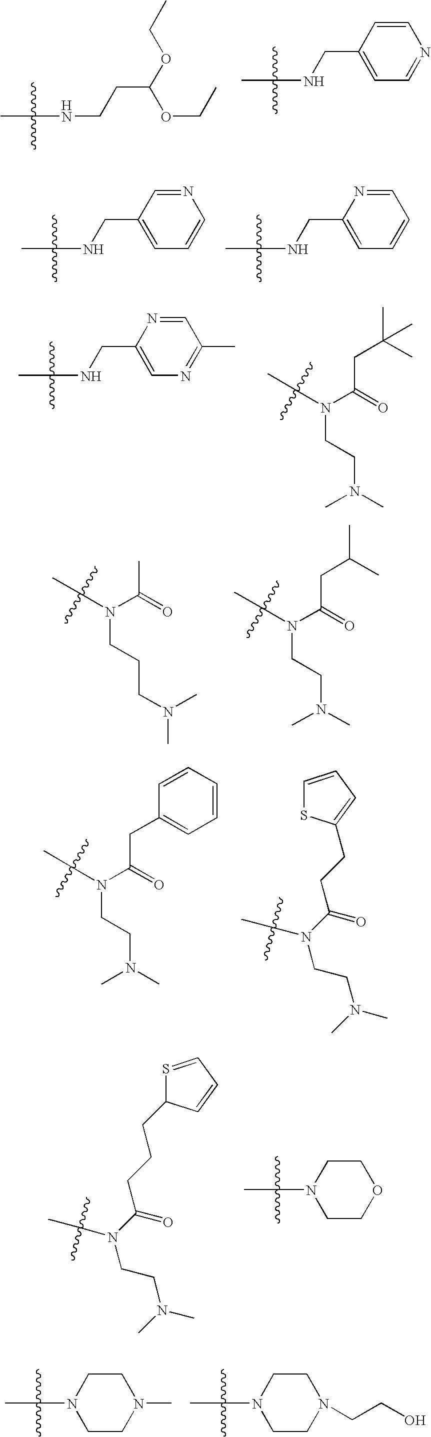

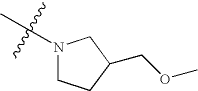

In another embodiment, R¹ is selected from the group of structures listed as Set 2b:

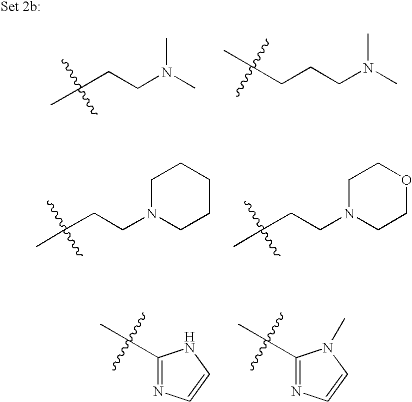

In one embodiment R⁶ is selected from the group consisting of hydrogen, methyl, alkyl, alkylcarbonyl, or alkoxycarbonyl. In another embodiment, R⁶ is hydrogen or methyl.

According to one embodiment, crystalline forms and polymorphs of the invention also includes any solvates and hydrates of the compounds of formulas I and II described.

Where present, compositions and crystalline polymorphs of the invention also include isomers either individually or as a mixture, such as enantiomers, diastereomers, and positional isomers.

Exemplary compounds of the present invention in the form of their corresponding free base include the following compounds:

| | |
|---|---|
| 1. | 4-{[4-(4-hydroxyphenyl)pyrimidin-2- |
| | yl]amino}benzenesulfonamide |
| 2. | N-[3-(dimethylamino)propyl]-4-[(4-{4-[2-(2- |
| | thienyl)ethoxy]phenyl}-pyrimidin-2- |
| | yl)amino]benzenesulfonamide |
| 3. | N-(3-(dimethylamino)propyl)-4-(4-(3-fluoro-4- |
| | methoxyphenyl)pyrimidin-2-ylamino)benzenesulfonamide |
| 4. | 4-(4-(4-(2-amino-3-phenylpropoxy)phenyl)pyrimidin-2- |
| | ylamino)benzenesulfonamide |
| 5. | 4-(4-(4-(2-amino-3-methylbutoxy)phenyl)pyrimidin-2- |
| | ylamino)benzenesulfonamide |
| 6. | N-(3-(dimethylamino)propyl)-4-(4-(6-fluoro-4- |
| | methoxyphenyl)pyrimidin-2-ylamino)benzene sulfonamide |
| 7. | N-(3-(dimethylamino)propyl)-4-(4-(2-fluoro-4- |
| | methoxyphenyl)pyrimidin-2-ylamino)benzene sulfonamide |
| 8. | N-(3-(dimethylamino)propyl)-4-(4-(5-fluoro-4- |
| | methoxyphenyl)pyrimidin-2-ylamino)benzene sulfonamide |
| 9. | N-(3-(dimethylamino)propyl)-4-(4-(3-trifluoromethyl-4- |
| | methoxyphenyl)pyrimidin-2-ylamino)benzenesulfonamide |

Exemplary crystalline polymorphs of the present invention include the following compounds of 3 as D-glucoronate salts:

| | |
|---|---|
| 10. | N-(3-(dimethylamino)propyl)-4-(4-(3-fluoro-4- |
| | methoxyphenyl)pyrimidin-2- |
| | ylamino)benzenesulfonamide D-glucoronate (Form I) |
| 11. | N-(3-(dimethylamino)propyl)-4-(4-(3-fluoromethyl-4- |
| | methoxyphenyl)pyrimidin-2- |
| | ylamino)benzenesulfonamide D-glucoronate (Form II) |
| 12. | N-(3-(dimethylamino)propyl)-4-(4-(3-fluoro-4- |
| | methoxyphenyl)pyrimidin-2- |
| | ylamino)benzenesulfonamide D-glucoronate (Form III) |
| 13. | N-(3-(dimethylamino)propyl)-4-(4-(3-fluoro-4- |
| | methoxyphenyl)pyrimidin-2- |
| | ylamino)benzenesulfonamide D-glucoronate (Form IV) |

According to one embodiment, the invention provides a method for preparing and manufacturing a crystalline form or polymorph of a compounds of formula I in the form of corresponding pharmaceutically acceptable salts, comprising the steps of: dissolving an amount of the compound of formula I and D-glucoronic acid, as a mixture or slurry; and precipitating one crystalline polymorph of the compound of formula I in the form of the D-glucoronic acid salt from the mixture or slurry. Precipitating refers to crystallization of the crystalline form or polymorph from the mixture or slurry, from the addition of a different solvent or solvents to the mixture or slurry, from concentration of the mixture or slurry, or from cooling the mixture or slurry.

According to a separate embodiment, the invention provides a method for preparing and manufacturing a crystalline form or polymorph of a compounds of formula I in the form of corresponding pharmaceutically acceptable salts, comprising the step of: recrystallizing one polymorph of the compound of formula I as the pharmaceutically acceptable salt, from one or more solvents diluted with water/solvents or from a different one or more solvents, to precipitate a different crystalline polymorph of the compound of formula I as the pharmaceutically acceptable salt.

According to a separate embodiment, the invention provides a method for preparing and manufacturing a crystalline form or polymorph of a compounds of formula I in the form of corresponding pharmaceutically acceptable salts, comprising the steps of: dissolving an amount of the compound of formula I and D-glucoronic acid, as a mixture or slurry; precipitating one crystalline polymorph of the compound of formula I in the form of the D-glucoronic acid salt from the mixture or slurry; and recrystallizing the precipitated one polymorph of the compound of formula I as the pharmaceutically acceptable salt, from one or more solvents diluted with water/solvents or from a different one or more solvents, to precipitate a different crystalline polymorph of the compound of formula I as the pharmaceutically acceptable salt.

In the embodiment, the amounts of the compound of formula I to the compounds selected from the group consisting of acids, bases and combinations thereof may be an equivalent amount by weight, an equivalent amount based on moles of reactants or an excess amount of the compound of formula I, based on equivalent weights (including weight ratios) or an excess amount of the compound, based on equivalent molar weight.

Compounds combined, mixed or slurried with the compounds of formula I are salt-forming compounds selected from acids, bases and combinations thereof. Suitable acids include organic acids and inorganic acids. Suitable organic acids include, but are not limited to for example, succinic acid, oxalic acid, acetic acid, D-, L-glucoronic acid, citric acid, malic acid, maleic acid, D-, L-glutamic acid, D-, L-tartaric acid and like organic acids.

According to an exemplary embodiment, equivalent molar amounts of D-glucoronic acid and the compound of N-(3-(dimethylamino)propyl)-4-(4-(3-fluoro-4-methoxyphenyl)pyrimidin-2-ylamino)benzenesulfonamide are combined to provide one crystalline form of the compound as a mono-D-glucoronate salt of formula:

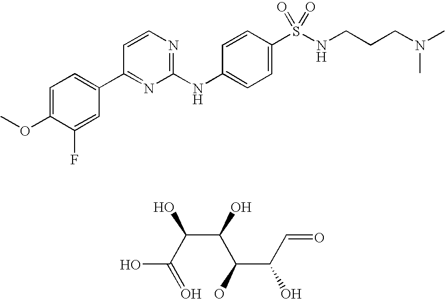

According to one embodiment, one polymorph of compound 3 as a D-glucoronate salt is prepared from D-glucoronic acid and compound 3 in a certain solvent mixture (e.g., isopropanol:water). The one crystalline polymorph precipitates and crystallizes out from the mixture of reactants and the one or more solvents. Recrystallizing the precipitated one crystalline polymorph of the compound 3 as the pharmaceutically acceptable D-glucoronate salt, from a different one or more solvents (e.g., acetone, t-buytlmethyl ether, isopropanol) or from diluting the one or more solvents with water/solvents, converts the one crystalline polymorph to a different crystalline polymorph of the compound 3 as the D-glucoronate salt, characterized by polycrystalline XRD.

According to a separate embodiment, the invention provides a method for converting one crystalline polymorph of the compound of formula I in the form of a pharmaceutically acceptable salt to a different crystalline polymorph of the compound of formula I as the pharmaceutically acceptable salt, comprising the steps of: heating an amount of one crystalline polymorph of the compound of formula I as the pharmaceutically acceptable salt to a temperature that converts it to a different crystalline polymorph of the compound of formula I as the pharmaceutically acceptable salt. The temperature needed to convert the one crystalline polymorph to a different crystalline polymorph depends on the thermal stability of the new polymorph relative to the one polymorph. Suitable temperatures for converting one crystalline polymorph to a different crystalline polymorph range from 40° C. to 250° C., including temperatures below the decomposition temperatures of particular crystalline polymorphs.

According to one embodiment, heating one crystalline polymorph of the compound of formula I in the form of a pharmaceutically acceptable salts converts the one polymorph to successively different crystalline polymorphs of the compound as the pharmaceutically acceptable salt, as each specific conversion range of higher temperatures is achieved. Different crystalline polymorphs are confirmed by analytical methods, including XRD patterns and data, DSC and TGA.

According to a separate embodiment, one crystalline polymorph of a compound of formula I as a pharmaceutically acceptable salt is converted to a different crystalline polymorph of the compound of formula I as the pharmaceutically acceptable salt by recrystallizing the one crystalline polymorph of a compound of formula I as a pharmaceutically acceptable salt using a combination of heating and one or more solvents.

Suitable solvents include water, mixtures of water and conventional organic solvents. Suitable organic solvents, include, but are not limited to for example, acetone, ethanol, methanol, ethyl ether, ethyl acetate, tetrahydrofuran (THF), dimethoxyothane, 1,3-dioxane, furan, ethylene glycol dimethyl ether, anisole, 1-propanol, 2-propanol, 2-methoxyethanol, ethylene glycol, 1-butanol, 2-butanol, diethylene, glycol, monoethyl ether, cyclohexanol, benzyl alcohol, phenol, glycerol, poly(ethylene glycol) (PEG), 2-nitroethanol, 2-fluoroethanol, 2,2,2-trifluoroethanol, ethylene glycol, 1-propanol, 2-propanol, 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, or glycerol. In some embodiments, the alcohol is ethanol. Suitable ethers include dimethoxymethane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, t-butyl methyl ether (TBME) and related solvents. The volume of water:solvent in the crystallizing solvent ranges from 1:1000 to 1000:1, including 1:10 to 100:1, and including 1:5 to 10:1.

Typically, water solubilities of the compounds in the form of their respective free base, such as compound 3, are low to none. In an exemplary embodiment, the solubility of the free base form of compound 3 in THF, DMSO and DMF was high but the solubility in group 3 solvents, or water for example, was low. For example, acetone is needed to dissolve the free base at 50° C. Solubility of the free base 3 in water was not detectable by a thermogravimeteric method (NiI). HCl, phosphoric acid, tartaric acid, acetic acid, D-glucoronic acid and succinic acid were used to prepare pharmaceutically acceptable salts of compound 3. One advantage of the D-glucoronate salt is its relatively high water solubility, 5.17 mg/mL, as compared to acetate (3.5 mg/mL) and succinate (1.1 mg/mL) salts of compound 3.

Preparations of crystalline polymorphs Form I-IV are provided in the Examples. In general, Form I can be prepared by dissolving compound 3 (any form, including amorphous) in a suitable solvent containing water and small amounts of solvent (e.g. isopropanol), precipitating the crystalline polymorph product from the solvent by any of numerous routine methods in the art such as by cooling or evaporating the solvent to induce precipitation. Suitable solvents include but are not limited to for example, water, a mixture of water and an alcohol, water and an ether, water mixture with conventional organic solvents and any suitable organic solvents. Water content of the solvent appears to influence the relative amounts of the Form I polymorph and the Form II polymorph, which precipitate from the mixture. Higher amounts of water in the solvent tend to favor the Form I polymorph, while lower amounts of water tend to favor the Form II polymorph. The Form II polymorph is converted back to the Form I polymorph by recrystallizing the Form II polymorph in acetone. The Form II polymorph is also used to prepare a different polymorph by adding it to TBME and recrystallizing to a Form III polymorph. The Form II polymorph is also used to prepare yet another different crystalline polymorph, a Form IV polymorph, by recrystallizing the Form II polymorph in isopropanol, converting the Form II polymorph to the Form IV polymorph.

In an exemplary embodiment, a weight ratio, based on molar weight, of D-glucoronic acid to a free base such as compound 3 is between 1:1 to 1:5. The solvent system also appears to play a role in converting of one polymorph to other polymorphs.

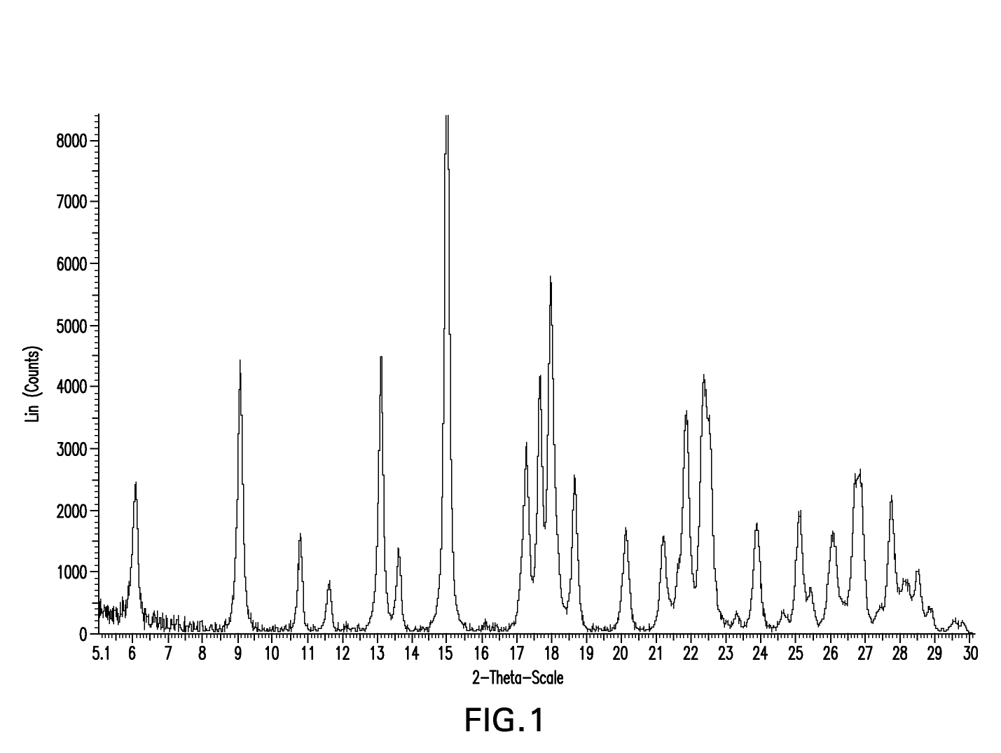

According to one embodiment, four crystalline polymorphs of compound 3 were isolated and characterized as D-glucoronate salts. The present invention provides one crystalline polymorph of N-(3-(dimethylamino)propyl)-4-(4-(3-fluoro-4-methoxyphenyl)pyrimidin-2-ylamino)benzenesulfonamide as a D-glucoronate, referred to herein as Form I. The Form I polymorph is identified by one or more solid-state analytical methods. The Form I polymorph exhibits a characteristic polycrystalline X-ray diffraction (XRD) pattern, as shown in FIG. 1. Polycrystalline XRD data consistent with the Form I polymorph are provided in Table 1 below. One skilled in the art would understand that the relative intensities of the polycrystalline XRD peaks can vary, depending upon the sample preparation technique, the sample mounting procedure, the particular instrument employed and sample particle size distribution. Moreover, instrument variation and other factors can affect the 2-theta values. Therefore, the polycrystalline XRD peak assignments can vary by plus or minus about 0.2°.

TABLE 1
X-RAY PEAK POSITIONS OF FORM I
D-GLUCORONATE OF N-(3-(DIMETHYLAMINO)PROPYL)-4-
(4-(3-FLUORO-4-METHOXYPHENYL)-PYRIMIDIN-2-
YLAMINO)BENZENESULFONAMIDE

| Angle, | d value, | Intensity, | Intensity |
|---|---|---|---|
| 2-Theta ° | Angstrom | Count | % |
| 6.0 | 14.6 | 2448.0 | 25.4 |
| 7.9 | 11.1 | 169.0 | 1.8 |
| 9.1 | 9.8 | 4433.0 | 46.0 |
| 10.8 | 8.2 | 1602.0 | 16.6 |
| 11.6 | 7.6 | 840.0 | 8.7 |
| 13.1 | 6.7 | 4475.0 | 46.4 |
| 13.6 | 6.5 | 1365.0 | 14.2 |
| 15.0 | 5.9 | 9642.0 | 100.0 |
| 17.3 | 5.1 | 3095.0 | 32.1 |
| 17.7 | 5.0 | 4142.0 | 43.0 |
| 18.0 | 4.9 | 5797.0 | 60.1 |
| 18.7 | 4.7 | 2551.0 | 26.5 |
| 20.2 | 4.4 | 1687.0 | 17.5 |
| 21.2 | 4.2 | 1556.0 | 16.1 |
| 21.9 | 4.1 | 3607.0 | 37.4 |
| 22.4 | 4.0 | 4193.0 | 43.5 |
| 22.6 | 3.9 | 3521.0 | 36.5 |
| 23.4 | 3.8 | 343.0 | 3.6 |
| 23.9 | 3.7 | 1763.0 | 18.3 |
| 24.7 | 3.6 | 337.0 | 3.5 |
| 25.1 | 3.5 | 1883.0 | 19.5 |
| 25.5 | 3.5 | 700.0 | 7.3 |
| 26.1 | 3.4 | 1626.0 | 16.9 |
| 26.8 | 3.3 | 2530.0 | 26.2 |
| 26.9 | 3.3 | 2629.0 | 27.3 |
| 27.8 | 3.2 | 2211.0 | 22.9 |
| 28.5 | 3.1 | 1021.0 | 10.6 |
| 28.9 | 3.1 | 402.0 | 4.2 |
| 29.7 | 3.0 | 214.0 | 2.2 |

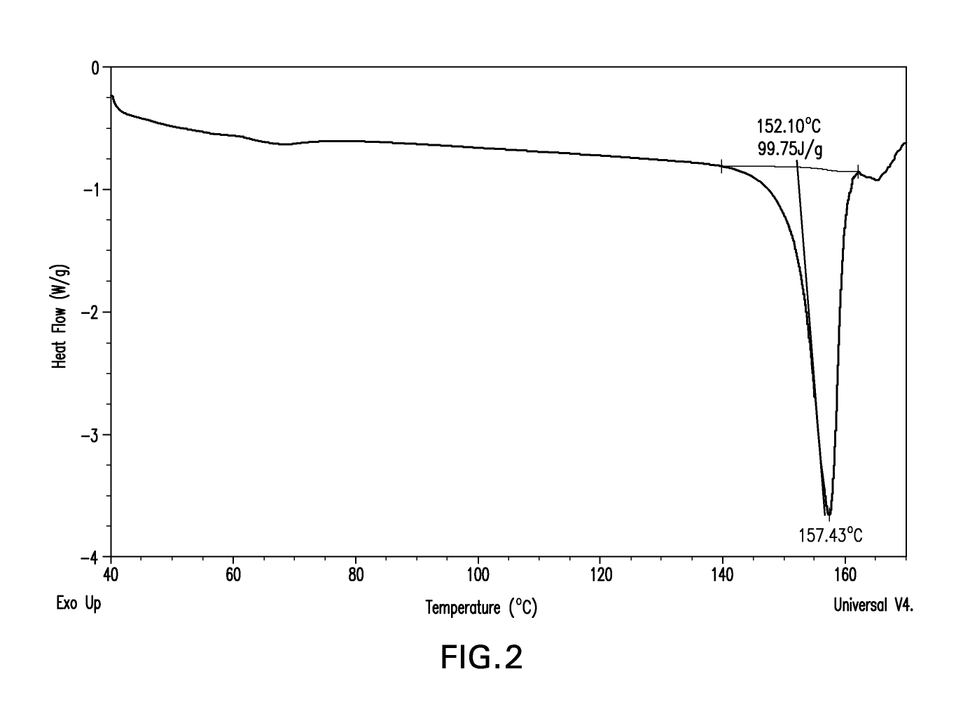

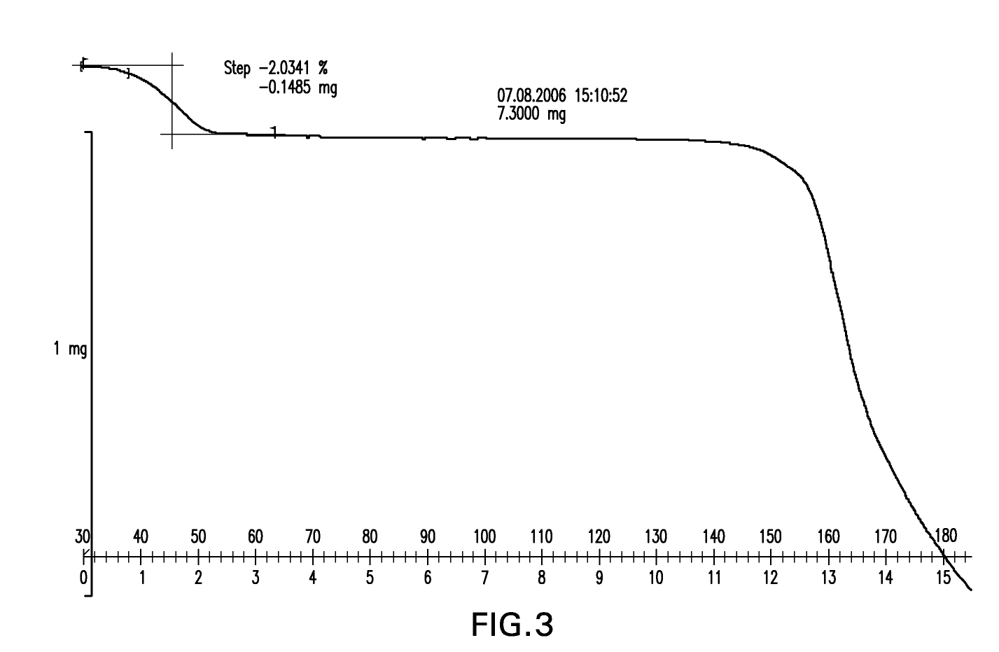

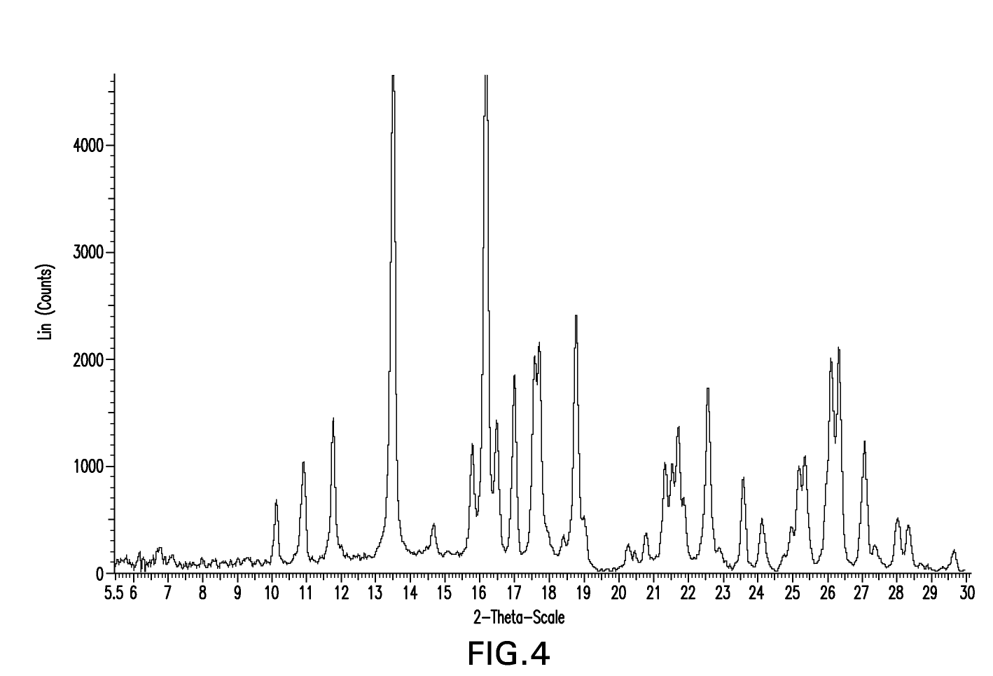

The present invention provides another crystalline polymorph of N-(3-(dimethylamino)propyl)-4-(4-(3-fluoro-4-methoxyphenyl)pyrimidin-2-ylamino)-benzenesulfonamide as a mono-D-glucuronate, referred to herein as Form II. The Form II polymorph is identified by one or more solid-state analytical methods. N-(3-(dimethylamino)propyl)-4-(4-(3-fluoro-4-methoxyphenyl)-pyrimidin-2-ylamino)-benzenesulfonamide as a D-glucoronate salt, is also identified by its characteristic differential scanning (DSC) trace, as shown in FIG. 2. Melting point is observed at 152.1° C., with an endotherm maxima at 157.4° C. N-(3-(dimethylamino)propyl)-4-(4-(3-fluoro-4-methoxyphenyl)pyrimidin-2-ylamino)benzenesulfonamide as a D-glucoronate salt, is also identified by its characteristic thermogravimetric analysis (TGA) thermogram, as shown in FIG. 3. A weight loss consistent with glucoronic acid is observed from the TGA thermogram. The Form II polymorph exhibits a characteristic polycrystalline XRD pattern, as shown in FIG. 4. Polycrystalline XRD data consistent with the Form II polymorph are provided in Table 2 below.

TABLE 2
X-RAY PEAK POSITIONS OF FORM II
D-GLUCORONATE OF N-(3-(DIMETHYLAMINO)PROPYL)-4-
(4-(3-FLUORO-4-METHOXYPHENYL)PYRIMIDIN-2-
YLAMINO)BENZENESULFONAMIDE

| Angle, | d value, | Intensity, | Intensity |
|---|---|---|---|
| 2-Theta ° | Angstrom | Count | % |
| 10.1 | 8.8 | 675 | 11.6 |
| 10.9 | 8.1 | 1031 | 17.8 |
| 11.8 | 7.5 | 1444 | 24.9 |
| 13.5 | 6.6 | 4843 | 83.5 |
| 14.7 | 6.0 | 449 | 7.7 |
| 15.8 | 5.6 | 1196 | 20.6 |
| 16.2 | 5.5 | 5800 | 100.0 |
| 16.5 | 5.4 | 1420 | 24.5 |
| 17.0 | 5.2 | 1843 | 31.8 |
| 17.6 | 5.0 | 2028 | 35.0 |
| 17.7 | 5.0 | 2146 | 37.0 |
| 18.8 | 4.7 | 2404 | 41.4 |
| 19.0 | 4.7 | 523 | 9.0 |
| 20.3 | 4.4 | 254 | 4.4 |
| 20.8 | 4.3 | 360 | 6.2 |
| 21.4 | 4.2 | 1021 | 17.6 |
| 21.6 | 4.1 | 1008 | 17.4 |
| 21.7 | 4.1 | 1358 | 23.4 |
| 21.9 | 4.1 | 688 | 11.9 |
| 22.6 | 3.9 | 1718 | 29.6 |
| 23.6 | 3.8 | 881 | 15.2 |
| 24.2 | 3.7 | 500 | 8.6 |
| 25.0 | 3.6 | 416 | 7.2 |
| 25.2 | 3.5 | 988 | 17.0 |
| 25.4 | 3.5 | 1079 | 18.6 |
| 26.2 | 3.4 | 1998 | 34.4 |
| 26.4 | 3.4 | 2107 | 36.3 |
| 27.1 | 3.3 | 1224 | 21.1 |
| 28.1 | 3.2 | 497 | 8.6 |
| 28.4 | 3.1 | 439 | 7.6 |
| 29.7 | 3.0 | 200 | 3.5 |

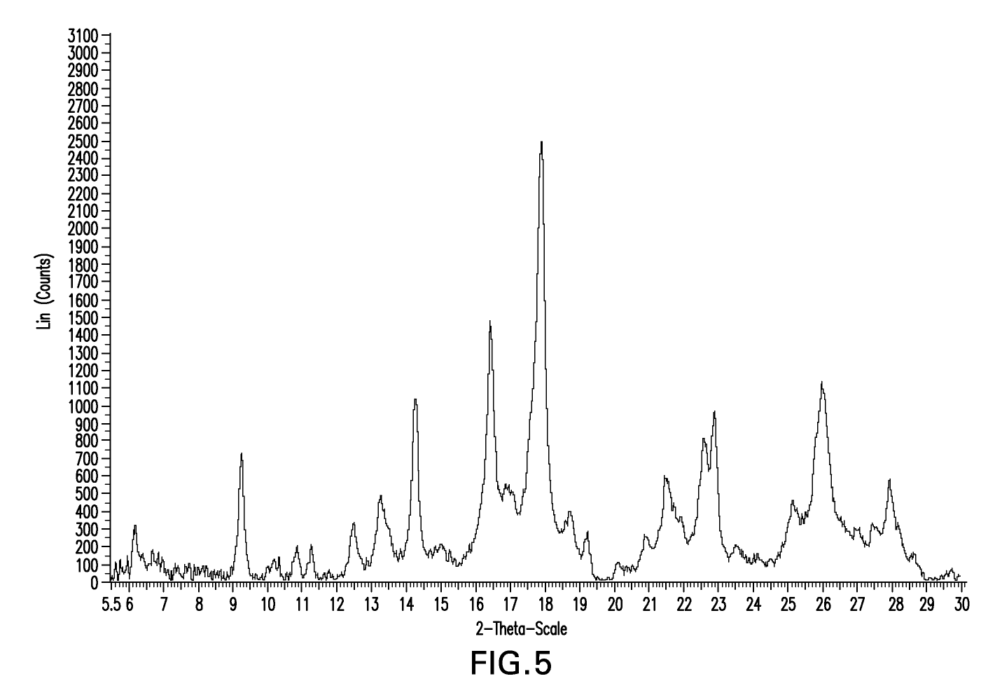

The present invention provides another crystalline polymorph of N-(3-(dimethylamino)propyl)-4-(4-(3-fluoro-4-methoxyphenyl)pyrimidin-2-ylamino)benzenesulfonamide as a D-glucuronate, referred to herein as Form III. The Form III polymorph is identified by one or more solid-state analytical methods. The Form III polymorph exhibits a characteristic powder X-ray diffraction (XRD) pattern, as shown in FIG. 5. Polycrystalline XRD data consistent with the Form III polymorph are provided in Table 3.

TABLE 3
X-RAY PEAK POSITIONS OF FORM III
D-GLUCORONATE OF N-(3-(DIMETHYLAMINO)PROPYL)-4-
(4-(3-FLUORO-4-METHOXYPHENYL)PYRIMIDIN-2-
YLAMINO)BENZENESULFONAMIDE

| Angle, | d value, | Intensity, | Intensity |
|---|---|---|---|
| 2-Theta ° | Angstrom | Count | % |
| 6.1 | 14.4 | 273 | 11.2 |
| 9.2 | 9.6 | 636 | 26.2 |
| 10.2 | 8.6 | 105 | 4.3 |
| 10.8 | 8.2 | 162 | 6.7 |
| 11.2 | 7.9 | 156 | 6.4 |
| 12.5 | 7.1 | 297 | 12.2 |
| 13.3 | 6.7 | 460 | 18.9 |
| 14.2 | 6.2 | 965 | 39.7 |
| 16.4 | 5.4 | 1365 | 56.2 |
| 17.9 | 5.0 | 2429 | 100.0 |
| 18.7 | 4.7 | 376 | 15.5 |
| 19.2 | 4.6 | 248 | 10.2 |
| 20.1 | 4.4 | 97.1 | 4.0 |
| 20.9 | 4.2 | 244 | 10.0 |
| 21.5 | 4.1 | 580 | 23.9 |
| 22.6 | 3.9 | 777 | 32.0 |
| 22.9 | 3.9 | 900 | 37.1 |
| 25.2 | 3.5 | 428 | 17.6 |
| 26.0 | 3.4 | 1086 | 44.7 |
| 28.0 | 3.2 | 528 | 21.7 |

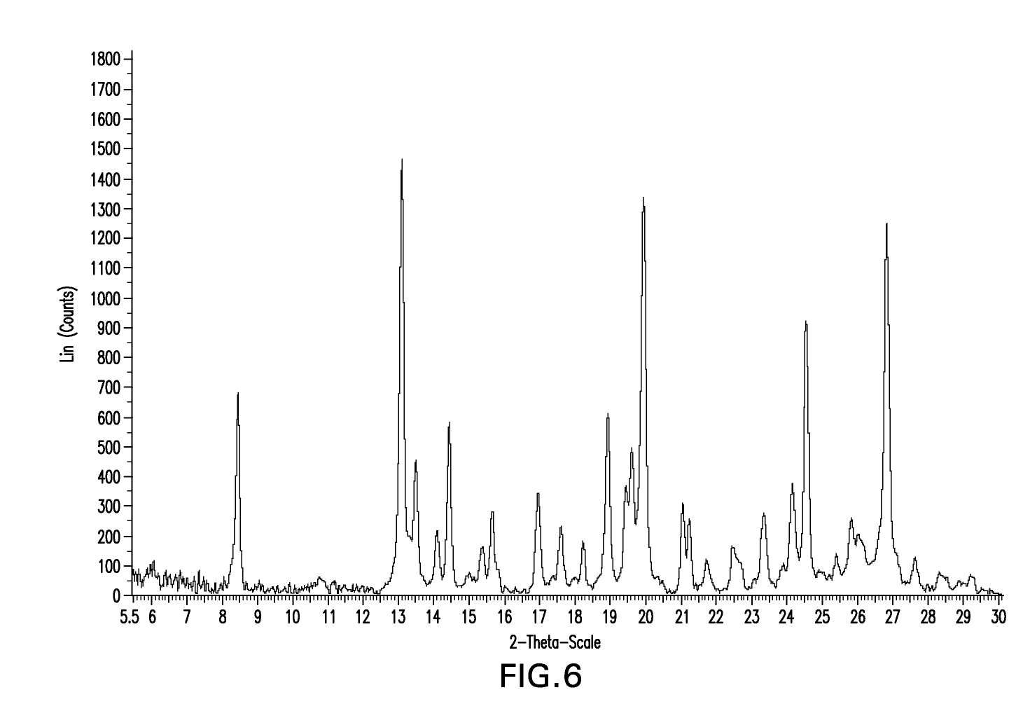

The present invention provides yet another crystalline polymorph of N-(3-(dimethylamino)propyl)-4-(4-(3-fluoro-4-methoxyphenyl)pyrimidin-2-ylamino)benzenesulfonamide as a D-glucuronate, referred to herein as Form IV. The Form IV polymorph is identified by one or more solid-state analytical methods. The Form IV polymorph exhibits a characteristic polycrystalline XRD pattern, as shown in FIG. 6. Polycrystalline XRD data consistent with the Form IV polymorph are provided in Table 4.

TABLE 4
X-RAY PEAK POSITIONS OF FORM IV
D-GLUCORONATE OF N-(3-(DIMETHYLAMINO)PROPYL)-4-
(4-(3-FLUORO-4-METHOXYPHENYL)PYRIMIDIN-2-
YLAMINO)BENZENESULFONAMIDE

| Angle, | d value, | Intensity, | Intensity |
|---|---|---|---|
| 2-Theta ° | Angstrom | Count | % |
| 8.4 | 10.5 | 677.0 | 46.2 |
| 13.1 | 6.8 | 1465.0 | 100.0 |
| 13.5 | 6.6 | 451.0 | 30.8 |
| 14.1 | 6.3 | 214.0 | 14.6 |
| 14.4 | 6.1 | 580.0 | 39.6 |
| 15.4 | 5.8 | 161.0 | 11.0 |
| 15.7 | 5.7 | 278.0 | 19.0 |
| 17.0 | 5.2 | 340.0 | 23.2 |
| 17.6 | 5.0 | 226.0 | 15.4 |
| 18.2 | 4.9 | 175.0 | 12.0 |
| 18.9 | 4.7 | 607.0 | 41.4 |
| 19.5 | 4.6 | 362.0 | 24.7 |
| 19.6 | 4.5 | 491.0 | 33.6 |
| 20.0 | 4.4 | 1338.0 | 91.3 |
| 21.1 | 4.2 | 305.0 | 20.8 |
| 21.3 | 4.2 | 251.0 | 17.1 |
| 21.7 | 4.1 | 115.0 | 7.8 |
| 22.5 | 3.9 | 160.0 | 10.9 |
| 23.4 | 3.8 | 274.0 | 18.7 |
| 24.2 | 3.7 | 373.0 | 25.4 |
| 24.6 | 3.6 | 920.0 | 62.8 |
| 25.9 | 3.4 | 256.0 | 17.5 |
| 26.9 | 3.3 | 1250.0 | 85.4 |

The presence of certain substituents in the compounds having formula I enable salts of the compounds to be formed. Suitable salts include pharmaceutically acceptable salts, for example acid addition salts derived from inorganic or organic acids, and salts derived from inorganic and organic bases. The phrase “pharmaceutically acceptable salt,” as used herein, is a salt formed from an acid and a basic nitrogen group of a pharmaceutically active agent. Illustrative salts include, but are not limited, to sulfate; citrate, acetate; oxalate; chloride; bromide; iodide; nitrate; bisulfate; phosphate; acid phosphate; isonicotinate; lactate; salicylate; acid citrate; tartrate; oleate; tannate; pantothenate; bitartrate; ascorbate; succinate; maleate; gentisinate; fumarate; D-, L-glucoronates; saccharate; formate; benzoate; glutamate; methanesulfonate; ethanesulfonate; benzenesulfonate; p-toluenesulfonate; pamoate (i.e., 1,1′-methylene-bis-(2-hydroxy-3-naphthoate)); and salts of fatty acids such as caproate, laurate, myristate, palmitate, stearate, oleate, linoleate, and linolenate salts.

The phrase “pharmaceutically acceptable salt” also refers to a salt prepared from a pharmaceutically active agent having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N,-dimethyl-N-(2-hydroxyethyl)amine, or tri-(2-hydroxyethyl)amine; N-methyl-D-glucamine; and amino acids such as arginine, lysine, and the like.

According to one embodiment an organic acid, D- or L-glucoronic acid was used to prepare polymorphic crystalline forms of formulas I and II. Other suitable acid addition salts include hydrochlorides, hydrobromides, hydroiodides, alkylsulphonates, e.g. methanesulphonates, ethanesulphonates, or isethionates, arylsulphonates, e.g. p-toluenesulphonates, besylates or napsylates, phosphates, sulphates, hydrogen sulphates, acetates, trifluoroacetates, propionates, citrates, maleates, fumarates, malonates, succinates, lactates, oxalates, tartrates and benzoates.

According to one embodiment, useful salts of compounds according to the invention include pharmaceutically acceptable salts, especially acid addition pharmaceutically acceptable salts.

Solid or crystalline forms or polymorphs of compounds having formula I can be achieved in the free base. One problem is that the free base typically has extremely low solubility in water, which lead to difficulties in preparing suitable dosage forms. The present invention provides methods for preparing and manufacturing polymorphs of crystalline salts of formula I having acceptable water solubility, which also improves bioavailability when ingested by a mammalian patient. As one example, the water solubility of the D-glucoronate salt of compound 3 is 5.7 mg/mL.

The invention also includes pharmaceutical compositions utilizing one or more of the present polymorphs along with one or more pharmaceutically acceptable carriers, excipients, additives and like agents.

Methods for preparing formulations including one or more crystalline forms and polymorphs of compounds of the present invention include the steps of preparing the compound as a crystalline salt, including polymorphs thereof, and formulating the salts with one or more pharmaceutically acceptable additives or carriers. Formulations of the invention provide effective amounts of a composition of the invention. Daily doses may range from about 0.1 mg to about 1000 mg for a person in need. Dose ranges may vary from about 10 mg/day to about 600 mg/day, including from 10 mg/day to about 60 mg/day. The dosing can be either in a single dose or two or more divided doses per day. Such doses can be administered in any manner that facilitates the compound's entry into the bloodstream including orally, via implants, parenterally (including intravenous, intraperitoneal, and subcutaneous injection), vaginally, rectally, and transdermally.

In some embodiments, formulations including salts and polymorphs of the invention are prepared and manufactured for administering transdermally, which includes all methods of administration across the surface of the body and the inner linings of body passages including epithelial and mucosal tissues. Such administering in certain embodiments includes, but is not limited to for example, a foam, a patch, a suspension, or a solution.

Oral formulations containing the salts and polymorphs of this invention can comprise any conventionally used oral forms, including tablets, capsules, buccal forms, troches, lozenges and oral liquids, suspensions or solutions. According to an exemplary embodiment, capsules may contain mixtures of one or more crystalline polymorphs in the desired percentage together with any other polymorph(s) of N-(3-(dimethylamino)propyl)-4-(4-(3-fluoro-4-methoxyphenyl)pyrimidin-2-ylamino)-benzenesulfonamide as a D-glucoronate salt or any structurally related compounds. Capsules or tablets of the desired crystalline form of the desired percentage composition may also be combined with mixtures of other active compounds or inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, and the like.

Tablet formulations can be additionally prepared and manufactured by conventional compression, wet granulation, or dry granulation methods and utilize pharmaceutically acceptable diluents (fillers), binding agents, lubricants, disintegrants, suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Oral formulations used herein can utilize standard delay or time-release formulations or spansules. Suppository formulations can be made from traditional materials, including cocoa butter, with or without the addition of waxes to alter the suppositories melting point, and glycerin. Water-soluble suppository bases, such as polyethylene glycols of various molecular weights, can also be used.

Excipient systems suitable for preparing formulations of the present inverted salts and polymorphs thereof include one or more fillers, disintegrants, and lubricants.

The filler component can be any filler component known in the art including, but not limited to, lactose, microcrystalline cellulose, sucrose, mannitol, calcium phosphate, calcium carbonate, powdered cellulose, maltodextrin, sorbitol, starch, or xylitol.

Disintegrants suitable for use in the present formulations of the invented salts and polymorphs thereof can be selected from those known in the art, including pregelatinized starch and sodium starch glycolate. Other useful disintegrants include croscarmellose sodium, crospovidone, starch, alginic acid, sodium alginate, clays (e.g. veegum or xanthan gum), cellulose floc, ion exchange resins, or effervescent systems, such as those utilizing food acids (such as citric acid, tartaric acid, malic acid, fumaric acid, lactic acid, adipic acid, ascorbic acid, aspartic acid, erythorbic acid, glutamic acid, and succinic acid) and an alkaline carbonate component (such as sodium bicarbonate, calcium carbonate, magnesium carbonate, potassium carbonate, ammonium carbonate, etc.). The disintegrant(s) useful herein can comprise from about 4% to about 40% of the composition by weight, preferably from about 15% to about 35%, more preferably from about 20% to about 35%.

The pharmaceutical formulations of the salts and polymorphs thereof and other structurally related compounds can also contain an antioxidant or a mixture of antioxidants, such as ascorbic acid. Other useful antioxidants include, but are not limited to for example, sodium ascorbate and ascorbyl palmitate, preferably in conjunction with an amount of ascorbic acid. An example range for the antioxidant(s) is from about 0.5% to about 15% by weight, most preferably from about 0.5% to about 5% by weight.

The formulations of the salts and polymorphs thereof described herein can be used in an uncoated or non-encapsulated solid form. In some embodiments, the pharmacological compositions are optionally coated with a film coating, for example, comprising from about 0.3% to about 8% by weight of the overall composition. Film coatings useful with the present formulations are known in the art and generally consist of a polymer (usually a cellulosic type of polymer), a colorant and a plasticizer. Additional ingredients such as wetting agents, sugars, flavors, oils and lubricants may be included in film coating formulations to impart certain characteristics to the film coat. The compositions and formulations herein may also be combined and processed as a solid, then placed in a capsule form, such as a gelatin capsule.

Pharmaceutical compositions of the salts and polymorphs thereof and other structurally related compounds and other structurally related compounds can be formulated with steroidal estrogens, such as conjugated estrogens. The amount used in the formulation can be adjusted according to the particular polymorph form or ratio of polymorph forms used, the amount and type of steroidal estrogen in the formulation as well as the particular therapeutic indication being considered. In general, the polymorphic composition ratio can be used in an amount sufficient to antagonize the effect of the particular estrogen to the level desired. The dose range of conjugated estrogens can be from about 0.3 mg to about 2.5 mg, about 0.3 mg to about 1.25 mg, or about 0.3 mg to about 0.625 mg. An example range for amount of N-(3-dimethylamino)propyl)-4-(4-(3-fluoro-4-methoxyphenyl)pyrimidin-2-ylamino)benzenesulfonamide as a mono-succinate salt or hemi-succinate salts in a combination formulation is about 10 mg to about 40 mg. For the steroidal estrogen mestranol, for example, a daily dosage can be from about 1 μg to about 150 μg, and for ethynyl estradiol a daily dosage of from about 1 μg to 300 μg can be used. In some embodiments, the daily dose is between about 2 μg and about 150 μg.

Diluents increase the bulk of a solid pharmaceutical composition, and may make a pharmaceutical dosage form containing the composition easier for the patient and caregiver to handle. Diluents for solid compositions include, for example, microcrystalline cellulose (e.g. Avicel™), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit™), potassium chloride, powdered cellulose, sodium chloride, sorbitol and talc.

Solid pharmaceutical compositions of the salts and polymorphs thereof are compacted into a dosage form, such as a tablet, which may include excipients whose functions include helping to bind the active ingredient and other excipients together after compression. Binders for solid pharmaceutical compositions include acacia, alginic acid, carbomer (e.g. Carbopol™), carboxymethylcellulose sodium, dextrin, ethyl cellulose, gelatin, guar gum, hydrogenated vegetable oil, hydroxyethyl cellulose, hydroxypropyl cellulose (e.g. Klucel™), hydroxypropyl methyl cellulose (e.g. Methocel™), liquid glucose, magnesium aluminum silicate, maltodextrin, methylcellulose, polymethacrylates, povidone (e.g. Kollidon™, Plasdone™), pregelatinized starch, sodium alginate, starch and others known in the art.

The dissolution rate of a compacted solid pharmaceutical composition including the salts and polymorphs thereof in the patient's stomach may be increased by the addition of a disintegrant to the composition. Disintegrants include alginic acid, carboxymethylcellulose calcium, carboxymethylcellulose sodium (e.g. Ac-Di-Sol™, Primellose™), colloidal silicon dioxide, croscarmellose sodium, crospovidone (e.g. Kollidon™, Polyplasdone™), guar gum, magnesium aluminum silicate, methyl cellulose, microcrystalline cellulose, polacrilin potassium, powdered cellulose, pregelatinized starch, sodium alginate, sodium starch glycolate (e.g. Explotab™), starch and others known in the art.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition including the salts and polymorphs thereof, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colorant to improve their appearance and/or facilitate patient identification of the product and unit dosage level.

In liquid pharmaceutical compositions including salts and polymorphs of the present invention, the compound of formula I and any other solid excipients are dissolved or suspended in a liquid carrier such as water, vegetable oil, alcohol, polyethylene glycol, propylene glycol or glycerin.

Liquid pharmaceutical compositions including salts and polymorphs of the invention may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl-cellulose, carbomer, cetostearyl alcohol and cetyl alcohol.

Liquid pharmaceutical compositions including salts and polymorphs of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition including salts and polymorphs of the invention may also contain a buffer such as gluconic acid, lactic acid, citric acid or acetic acid, sodium gluconate, sodium lactate, sodium citrate or sodium acetate. Selection of excipients and the amounts used may be readily determined by the formulation scientist based upon experience and consideration of standard procedures and reference works in the field.

The solid compositions including salts and polymorphs of the present invention include powders, granulates, aggregates and compacted compositions. The dosages include dosages suitable for oral, buccal, rectal, parenteral (including subcutaneous, intramuscular, and intravenous), inhalant and ophthalmic administration. The most suitable administration in any given case will depend on the nature and severity of the condition being treated. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions and elixirs. The dosage form of the present invention may be a capsule containing the composition, such as a powdered or granulated solid composition of the invention, within either a hard or soft shell. The shell may be made from gelatin and optionally contain a plasticizer such as glycerin and sorbitol, and an opacifying agent or colorant. The active ingredient of the invention (salts and polymorphs thereof) and excipients may be formulated into compositions and dosage forms according to methods known in the art.

A composition, including salts and polymorphs of the invention, for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition, including salts and polymorphs of the invention, may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling including salts and polymorphs of the present invention may include any of the aforementioned blends and granulates that were described with reference to tableting, however, they are not subjected to a final tableting step.

Methods of administering a pharmaceutical composition including salts and polymorphs of the invention are not specifically restricted, and can be administered in various preparations depending on the age, sex, and symptoms of the patient. For example, tablets, pills, solutions, suspensions, emulsions, granules and capsules may be orally administered. Injection preparations may be administered individually or mixed with injection transfusions such as glucose solutions and amino acid solutions intravenously. If necessary, the injection preparations are administered singly intramuscularly, intracutaneously, subcutaneously or intraperitoneally. Suppositories may be administered into the rectum.

The amount of the compound of formulas I and II in the form of salts and polymorphs thereof contained in a pharmaceutical composition according to the present invention is not specifically restricted, however, the dose should be sufficient to treat, ameliorate, or reduce the targeted symptoms. The dosage of a pharmaceutical composition according to the present invention will depend on the method of use, the age, sex, and condition of the patient.

The pharmaceutical compositions including salts and polymorphs of the present invention may comprise the compound of the present invention or in combination with other kinase-inhibiting compounds or chemotherapeutic agents. Chemotherapeutic agents include, but are not limited to exemestane, formestane, anastrozole, letrozole, fadrozole, taxane and derivatives such as paclitaxel or docetaxel, encapsulated taxanes, CPT-11, camptothecin derivatives, anthracycline glycosides, e.g., doxorubicin, idarubicin, epirubicin, etoposide, navelbine, vinblastine, carboplatin, cisplatin, estramustine, celecoxib, tamoxifen, raloxifen, Sugen SU-5416, Sugen SU-6668, and Herceptin.

Having described the invention, the invention is further illustrated by the following non-limiting examples.

### EXAMPLES

Acquisition of analytical data. Differential scanning calorimetry (DSC) data were collected on pharmaceutically acceptable D-gluconate salts of compound 3 using a DSC (TA instruments, model Q1000) under the following parameters: 50 mL/min. purge gas (N₂); scan range 40 to 200° C., scan rate 10° C./min. Thermo-gravimetric analysis data was collected using a TGA instrument (Mettler Toledo, model TGA/SDTA 851e) under the following parameters: 40 mL/min. purge gas (N₂); scan range 30 to 250° C., scan rate 10° C./min. X-Ray data was acquired using an X-ray powder diffractometer (Bruker-axs, model D8 advance) having the following parameters: voltage 40 kV, current 40.0 mA, scan range (2θ) 5 to 300, scan step size 0.01°, VANTEC detector, and anti-scattering slit 1 mm.

### Example 1

### Form I Polymorph of the D-glucoronate salt of N-(3-(dimethylamino)propyl)-4-(4-(3-fluoro-4-methoxyphenyl)pyrimidin-2-ylamino)-benzenesulfonamide

The free base compound 3 (65.9 mg) was poured into vial and 0.66 mL water was added at room temperature. The solution of compound 3 was combined with D-glucoronic acid (29.5 mg) and 0.77 mL of isoproponal was added. The mixture was stirred for 1 hour and then allowed to form a crystalline precipitate. The mono-D-glucoronate salt of compound 3 was characterized by a combination of analytical techniques. The melting point of the D-glucoronate salt of compound 3 is 152° C. from the endotherm indicated by DSC analysis. The ratio of D-glucoronate to compound 3 was determined to be 1:1. TGA indicated a weight loss consistent with glucoronic acid. XRD data for the crystalline form is summarized in Table 1.

### Example 2

### Form II Polymorph of D-glucoronate salt of N-(3-(dimethylamino)propyl)-4-(4-(3-fluoro-4-methoxyphenyl)pyrimidin-2-ylamino)benzene-sulfonamide

The free base compound 3 (857.9 mg) was poured into vial and 6.86 mL water was added at room temperature. The solution of compound 3 was combined with D-glucoronic acid (378 mg) and 0.30 mL of water and 6.56 mL isopropanol was added. The mixture was stirred for 48 hours and then allowed to form a crystalline precipitate. The crystals were filtered and dried at 50° C. under vacuum. The D-glucoronate salt of compound 3 was characterized by a combination of analytical techniques. The melting point of the D-glucoronate salt of compound 3 is 152° C. from the endotherm indicated by DSC analysis. The ratio of D-glucoronate to compound 3 was determined to be 1:1. TGA indicated a weight loss consistent with glucoronic acid. XRD data for the crystalline Form II polymorph is summarized in Table 2.

### Example 3

### Conversion of Form II Polymorph of the D-glucoronate salt of N-(3-(dimethylamino)propyl)-4-(4-(3-fluoro-4-methoxyphenyl)-pyrimidin-2-ylamino)benzene-sulfonamide to Form I Polymorph of D-glucoronate salt of N-(3-(dimethylamino)propyl)-4-(4-(3-fluoro-4-methoxyphenyl)-pyrimidin-2-ylamino)benzene-sulfonamide

The Form II polymorph of compound 3 as the D-glucoronate salt was slurried in acetone at room temperature for 2 days to produce the Form I polymorph of compound 3 as a D-glucoronate salt, as confirmed by comparing XRD data with the Form I polymorph in Table I.

### Example 4

### Form III Polymorph of the D-glucoronate salt of N-(3-(dimethylamino)propyl)-4-(4-(3-fluoro-4-methoxyphenyl)pyrimidin-2-ylamino)-benzenesulfonamide

The Form II polymorph of compound 3 as a D-glucoronate salt was slurried in t-BME at room temperature for 4 days and then allowed to form a crystalline precipitate. The crystals were filtered and dried at 50° C. under vacuum. The D-glucoronate salt of compound 3 was characterized by a combination of analytical techniques. XRD data for the crystalline Form III polymorph is summarized in Table 3.

### Example 5

### Form IV Polymorph of D-glucoronate salt of N-(3-(dimethylamino)propyl)-4-(4-(3-fluoro-4-methoxyphenyl)pyrimidin-2-ylamino)-benzenesulfonamide

The Form II polymorph of compound 3 as a D-glucoronate salt was slurried in isopropanol at room temperature for 4 days and then allowed to form a crystalline precipitate. The crystals were filtered and dried at 50° C. under vacuum. The D-glucuronate salt of compound 3 was characterized by a combination of analytical techniques. XRD data for the crystalline Form IV polymorph is summarized in Table 4.

What is claimed is:

1. A method for manufacturing a crystalline polymorph of a compound of formula I: 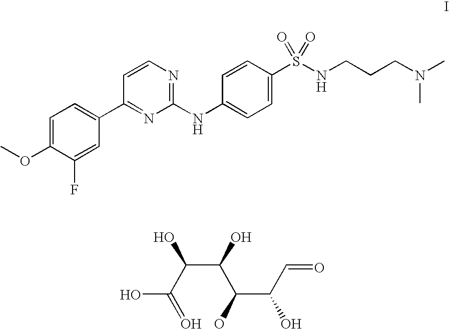
comprising the steps of: dissolving an amount of a compound of N-(3-(dimethylamino)propyl)-4-(4-(3-fluoro-4-methoxyphenyl)-pyrimidin-2-ylamino)-benzenesulfonamide and D-glucoronic acid, as a mixture, in one or more solvents; and precipitating one crystalline polymorph of the compound of formula I as the D-glucoronate salt from the mixture.

2. A method for manufacturing a crystalline polymorph of a compound of formula I: 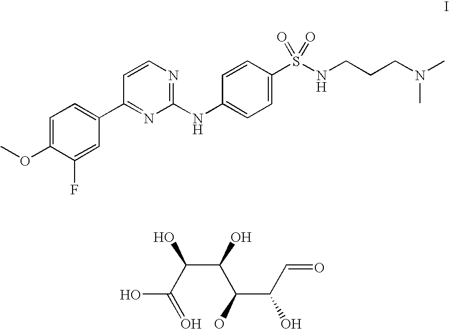
comprising the step of: recrystallizing one crystalline polymorph of the compound of formula I, from one or more solvents to precipitate a different crystalline polymorph of the compound of formula I.

3. The method of claim 1, further comprising a mixture of N-(3-(dimethylamino)propyl)-4-(4-(3-fluoro-4-methoxyphenyl)-pyrimidin-2-ylamino)-benzenesulfonamide and D-glucoronic acid in amounts, based on molar weight equivalents, of from 1:1 to 1:5.

4. The method of claim 3, wherein the one or more solvents is a water:isopropanol mixture.

5. The method of claim 4, wherein the D-glucoronate salt is characterized by X-ray diffraction peaks at the following angles (±0.2°) of 2θ in its X-ray diffraction pattern: 6.0°, 9.1°, 10.8°, 13.1°, 13.6°, 15.0°, 17.3°, 17.7°, 18.0°, 18.7°, 20.2°, 21.2°, 21.9°, 22.4°, 22.6, 23.9, 25.1°, 26.1°, 26.8°, 26.9°, 27.8° and 28.5°.

6. The method of claim 3, wherein the one or more solvents is a water:isopropanol mixture.

7. The method of claim 6, wherein the D-glucoronate salt characterized by X-ray diffraction peaks at the following angles (±0.2°) of 2θ in its X-ray diffraction pattern: 10.1°, 10.9°, 11.8°, 13.5°, 15.8°, 16.2°, 16.5°, 17.0°, 17.6°, 18.8°, 21.4°, 21.6°, 21.7°, 22.6°, 23.6, 25.2, 26.2°, 26.4° and 27.1°.

8. The method of claim 3, wherein the one or more solvents is tertiary-butyl methyl ether (TBME).

9. The method of claim 8, wherein the D-glucoronate salt characterized by X-ray diffraction peaks at the following angles (±0.2°) of 2θ in its X-ray diffraction pattern: 6.1°, 9.2°, 12.5°, 13.3°, 14.2°, 16.4°, 17.9°, 18.7°, 21.5°, 22.6°, 22.9°, 25.2°, 26.0° and 28.0°.

10. The method of claim 2, wherein the one or more solvents is isopropanol.

11. The method of claim 10, wherein the D-glucoronate salt characterized by X-ray diffraction peaks at the following angles (±0.2°) of 2θ in its X-ray diffraction pattern: 8.4°, 13.1°, 13.5°, 14.1°, 14.4°, 15.7°, 17.0°, 17.6°, 18.2°, 19.5°, 19.6°, 20.0°, 21.1°, 21.3°, 22.5, 23.4, 24.2°, 24.6°, 25.9° and 26.9°.

12. The method of claim 2, wherein the one or more solvents is acetone.

13. The method of claim 12, wherein the D-glucoronate salt characterized by X-ray diffraction peaks at the following angles (±0.2°) of 2θ in its X-ray diffraction pattern: 6.0°, 9.1°, 10.8°, 13.1°, 13.6°, 15.0°, 17.3°, 17.7°, 18.0°, 18.7°, 20.2°, 21.2°, 21.9°, 22.4°, 22.6, 23.9, 25.1°, 26.1°, 26.8°, 26.9°, 27.8° and 28.5°.

14. The method of claim 1, wherein the polymorph exhibits an endotherm at 152° C. from differential scanning calorimetry.

15. The method of claim 1, wherein the D-glucoronate salt has a water solubility of 5.17 mg/mL.

16. A method for converting one crystalline polymorph of a compound of formula I to one or more different polymorphs of the compound of formula I: 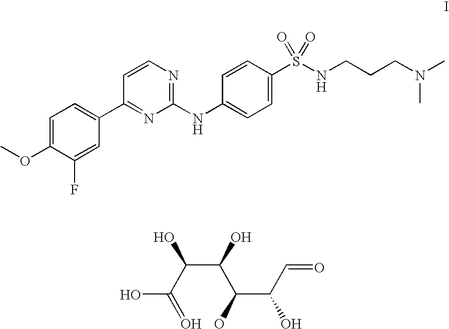
comprising the steps of: dissolving an amount of a compound of N-(3-(dimethylamino)propyl)-4-(4-(3-fluoro-4-methoxyphenyl)-pyrimidin-2-ylamino)-benzenesulfonamide and D-glucoronic acid, as a mixture, in one or more solvents; precipitating one crystalline polymorph of the compound of formula I as the D-glucoronate salt from the mixture; and recrystallizing the precipitated one crystalline polymorph of the compound of formula I, from the one or more solvents or from a different one or more solvents, converting the one crystalline polymorph of the compound of formula I as the D-glucoronate salt to a different crystalline polymorph of the compound of formula I.

17. The method of claim 16, wherein a crystalline Form II polymorph of the compound of formula I is converted to a crystalline Form I polymorph of the compound of formula I by recrystallizing the Form II polymorph of the compound of formula I in acetone.

18. The method of claim 16, wherein one crystalline polymorph of the compound of formula I as the D-glucoronate salt is recrystallized from tertiary-butyl methyl ether (TBME).

19. The method of claim 18, wherein the mono-D-glucoronate salt is characterized by X-ray diffraction peaks at the following angles (±0.2°) of 2θ in its X-ray diffraction pattern: 6.1°, 9.2°, 12.5°, 13.3°, 14.2°, 16.4°, 17.9°, 18.7°, 21.5°, 22.6°, 22.9°, 25.2°, 26.0° and 28.0°.

20. The method of claim 16, wherein one crystalline polymorph of the compound of formula I as the D-glucoronate salt is recrystallized from isopropanol.

21. The method of claim 20, wherein the D-glucoronate salt is characterized by X-ray diffraction peaks at the following angles (±0.2°) of 2θ in its X-ray diffraction pattern: 8.4°, 13.1°, 13.5°, 14.1°, 14.4°, 15.7°, 17.0°, 17.6°, 18.2°, 19.5°, 19.6°, 20.0°, 21.1°, 21.3°, 22.5, 23.4, 24.2°, 24.6°, 25.9° and 26.9°.

22. A method for converting one crystalline polymorph of a compound of formula I to one or more different polymorphs of the compound of formula I: 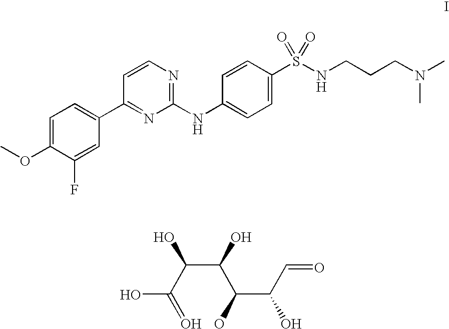
comprising the steps of: heating an amount of one polymorph of the compound of formula I to a temperature that converts the one crystalline polymorph of the compound of formula I to a different polymorph of the compound of formula I as the pharmaceutically acceptable salt.

23. The method of claim 22, further comprising a temperature ranging from 40° to 250° C.

24. A method for manufacturing a pharmaceutical composition comprising the step of: combining a compound of formula I according to claim 1, or in combination with other kinase-inhibiting pharmaceutical compositions or chemotherapeutic agents, and a pharmaceutically acceptable carrier.

25. A method for manufacturing a pharmaceutical composition comprising the step of: combining a compound of formula I according to claim 2, or in combination with other kinase-inhibiting pharmaceutical compositions or chemotherapeutic agents, and a pharmaceutically acceptable carrier.

26. A method for manufacturing a pharmaceutical composition comprising the step of: a compound of formula I according to claim 16, or in combination with other kinase-inhibiting pharmaceutical compositions or chemotherapeutic agents, and a pharmaceutically acceptable carrier.

27. A method of inhibiting kinase activity in a mammal comprising administering to a mammal a kinase-inhibiting amount of a compound of formula I manufactured according to claim 1.

28. The method of claim 27, wherein the mammal is a human.

29. A method of inhibiting kinase activity in a mammal comprising administering to a mammal a kinase-inhibiting amount of a pharmaceutical compound manufactured according to claim 24.

30. A method of treating a kinase-dependent condition comprising administering to a subject a kinase-inhibiting amount of a pharmaceutical composition manufactured according to claim 25.

31. A method of treating a kinase-dependent condition comprising administering to a subject a kinase-inhibiting amount of a pharmaceutical composition manufactured according to claim 26.
